(19) United States
(12) Patent Application Publication
(10) Pub. No.: US 2020/0253254 A1
(43) Pub. Date: Aug. 13, 2020

(54) MEAT-MODIFYING COMPOSITION

(71) Applicant: AJINOMOTO CO., INC., Tokyo (JP)

(72) Inventors: Ryusuke KOBA, Kawasaki-shi (JP); Soichiro KORI, Kawasaki-shi (JP)

(73) Assignee: AJINOMOTO CO., INC., Tokyo (JP)

(21) Appl. No.: 16/863,087

(22) Filed: Apr. 30, 2020

Related U.S. Application Data
(63) Continuation of application No. PCT/JP2018/041580, filed on Nov. 9, 2018.

(30) Foreign Application Priority Data
Nov. 9, 2017 (JP) 2017-216796

(51) Int. Cl. A23L 13/70; C12N 9/34

(52) U.S. Cl. CPC A23L 13/74; A23V 2002/00; C12Y 302/01003; C12N 9/2428

(57) ABSTRACT

Compositions that contain gluconate and a saccharide-modifying enzyme at a proportion of 100 to 100,000,000 U of the saccharide-modifying enzyme per 1 g of gluconic acid can be used to provide a juicy and tender prepared meat product with suppressed syneresis by heating.

### CROSS REFERENCES TO RELATED APPLICATIONS

This application is a continuation of International Patent Application No. PCT/JP2018/041580, filed on Nov. 9, 2018, and claims priority to Japanese Patent Application No. 2017-216796, filed on Nov. 9, 2017, both of which are incorporated herein by reference in their entireties.

### BACKGROUND OF THE INVENTION

### Field of the Invention

The present invention relates to compositions for modifying meat, prepared meat products containing such a composition, and production methods thereof.

### Discussion of the Background

In the meat processing field and meat cooking field, meat is cut and subjected to various treatments. Among them, when a heat treatment such as boiling, baking, frying or the like is applied to meat, it is known that meat loses a juicy feeling and the texture is degraded due to denaturation by heating.

To solve these problems, some attempts have been made. As exemplary use of organic acid salts and the like, a meat treating agent using an alkali metal salt of gluconic acid for improving the quality of boiled meat (see JP-B-4203555, which is incorporated herein by reference in its entirety), a preparation containing gluconate, calcium lactate and an alkali agent for treating marine products or meat (see JP-B-5882792, which is incorporated herein by reference in its entirety), and a processed meat improving agent containing oligosaccharide, a starchy raw material and a soluble salt of an organic acid (see JP-A-2006-67998, which is incorporated herein by reference in its entirety) have been reported. As exemplary use of enzyme, a food quality improver containing a starch degrading enzyme and protease in combination and showing a softening action on meat and the like (see JP-A-7-31396, which is incorporated herein by reference in its entirety), a meat quality and flavor improver containing protease, amylase and the like (see JP-A-5-276899, which is incorporated herein by reference in its entirety), and a production method of a processed meat and the like with improved properties and taste which uses an enzyme having a transglucosylation activity, transglutaminase and the like (see JP-B-5056193, which is incorporated herein by reference in its entirety) have been reported.

### SUMMARY OF THE INVENTION

Accordingly, it is one object of the present invention to suppress syneresis due to heating of meat.

It is another object of the present invention to enhance the water-holding property of meat after heating.

It is another object of the present invention to provide a processed meat product with improved juiciness.

These and other objects, which will become apparent during the following detailed description, have been achieved by the inventors' discovery that syneresis due to a heat treatment is suppressed and a prepared meat product with high water-holding property and a superior, juicy and tender texture can be obtained by bringing the meat in contact with a composition containing gluconate and a saccharide-modifying enzyme.

That is, the present invention provides the following.

(1) A composition for modifying meat, comprising gluconate and a saccharide-modifying enzyme.

(2) The composition of (1), wherein the saccharide-modifying enzyme is contained at 100 to 100,000,000 U per 1 g of gluconic acid.

(3-1) The composition of (1) or (2), wherein the saccharide-modifying enzyme is glucoamylase.

(3-2) The composition of (1) or (2), wherein the saccharide-modifying enzyme is α-glucosidase.

(4) The composition of any of (1) to (3), wherein the gluconate is at least one selected from the group consisting of sodium gluconate, potassium gluconate and calcium gluconate.

(5) A method for producing a prepared meat product, comprising a step of bringing meat in contact with gluconate and a saccharide-modifying enzyme.

(6-1) The method of (5), wherein the saccharide-modifying enzyme is glucoamylase.

(6-2) The method of (5), wherein the saccharide-modifying enzyme is α-glucosidase.

(7) The method of (5) or (6), wherein the gluconate is comprised such that 0.001 to 5 g of gluconic acid is contacted per 100 g of the meat.

(8) The method of any of (5) to (7), wherein 0.1 to 500,000,000 U of the saccharide-modifying enzyme is contacted per 100 g of the meat.

(9) A prepared meat product comprising gluconate and a saccharide-modifying enzyme, wherein the gluconate is comprised such that 0.001 to 5 g of gluconic acid is contained, and 0.1 to 500,000,000 U of the saccharide-modifying enzyme is contained, each per 100 g of meat.

(10) A method for modifying meat, comprising a step of bringing the meat in contact with gluconate and a saccharide-modifying enzyme.

### Effect of the Invention

According to the present invention, a prepared meat product that does not lose umami or juiciness of the meat even after heating and can afford a juicy and moist texture can be provided.

### DETAILED DESCRIPTION OF THE PREFERRED EMBODIMENTS

The present invention relates to a composition containing gluconate and a saccharide-modifying enzyme for modifying meat (hereinafter sometimes to be abbreviated as the composition of the present invention).

The gluconate to be used in the present invention is, for example, a pharmacologically acceptable salt of gluconic acid. Examples thereof include alkali metals such as lithium, sodium, potassium and the like; alkaline earth metals such as calcium, magnesium and the like; ammonium; and salts with basic organic substance and the like and salts with minerals such as iron, copper, manganese and the like. Among these, from the aspect of salt generally used for foods, salts with sodium, potassium and calcium are preferable, sodium and potassium are more preferable, and a salt with sodium is further preferable.

In addition, gluconate may be a commercially available one or one obtained by a method known per se.

Furthermore, gluconate may be any of a hydrate, a non-hydrate, a non-solvate and a solvate.

Saccharide-modifying enzymes are classified into enzymes having glycolytic activity, enzymes having glycosyltransferase activity, and enzymes that oxidize saccharide. The saccharide-modifying enzyme used in the present invention is not particularly limited as long as the effect of the present invention is not inhibited. Particularly, from the aspects of suppression of syneresis due to heat treatment and improvement of juicy feeling by high water retention, an enzyme having a glycolytic activity (hereinafter to be abbreviated as a glycolytic enzyme) that hydrolyzes a glycoside bond of saccharide is preferable.

In the present invention, the glycolytic enzyme includes α-amylase, β-amylase, invertase, maltotriohydrolase, pullulanase, amyloglucosidase, α-glucosidase, β-glucosidase, isoamylase, glucoamylase, pectinase, cellulase, hemicellulase and the like. Among these, glucoamylase and α-glucosidase, which are exo-type enzymes that successively decompose α-1,4 glycoside bond in glucose units, are preferable, and glucoamylase is more preferable.

The origin of the saccharide-modifying enzyme is not particularly limited, and may be any of plants, animals, microorganisms, and the like. Specific examples include those derived from filamentous fungi, preferably those derived from the genus Aspergillus, more preferably those derived from Aspergillus niger. Also, the enzyme may be a recombinant enzyme produced by genetic engineering technology. Only one kind of the saccharide-modifying enzyme or two or more kinds thereof may be used in combination, and one obtained by a method known per se or a commercially available one may be used.

In the present invention, the form of meat is not particularly limited and, for example, block meat, sliced meat (thinned meat, thickly sliced meat, thinly sliced meat), small sliced meat, minced meat, sliced fish and the like can be mentioned, and block meat, sliced meat (thin sliced meat, thickly sliced meat, thinly sliced meat), and small sliced meat are preferable. The size and shape of the meat are not particularly limited.

The meat kind is not particularly limited and, for example, meat and poultry such as pig, cow, chicken, sheep, goat, horse, camel, bear, rabbit, wild duck, pigeon, duck, quail, alpaca, and the like; fish such as salmon, salmon, cod, sea bream, tuna, swordfish, bonito, sardines, and the like; and the like can be mentioned. Particularly, since the effect of the present invention is more easily afforded, meat and poultry such as pig, cow, chicken, sheep, goat, horse, rabbit, wild duck and the like are preferable, and pig, cow, and chicken are more preferable.

In the present invention, the “meat modification” refers to improving or enhancing the quality of meat after heat cooking, such as texture, taste, flavor and the like.

The texture is a feeling such as chewiness and feeling on the tongue when food is put into the mouth. In the case of meat products, it refers to a juicy feeling, tenderness, elasticity, crispness, fibrousness, fleshiness, dryness, and the like.

The proportion of gluconate and a saccharide-modifying enzyme contained in the composition of the present invention is, when converted to gluconic acid, such that the enzyme activity of the saccharide-modifying enzyme is generally 100 to 100,000,000 U, preferably 500 to 50,000,000 U, more preferably 2,000 to 20,000,000 U, per 1 g of gluconic acid, from the aspects of quality such as texture, taste, flavor and the like.

As for the enzyme activity of the saccharide-modifying enzyme in the present specification, the amount of the enzyme that achieves an increase in the absorption at wavelength of 400 to 420 nm that corresponds to 1 μmol of p-nitrophenol using 4-nitrophenyl α-D-glucopyranoside or block p-nitrophenyl maltoheptaside (BPNPG7) as a substrate per one minute at 40±0.5° C. is defined as 1 unit (1 U).

The composition of the present invention may be in a form concurrently containing a saccharide-modifying enzyme and gluconate, or in the form of a kit for combining, immediately before use, the separately-prepared components. From the aspects of workability and convenience, the form of concurrently containing them is preferable.

The composition of the present invention may contain other components as long as the effect of the present invention is not impaired. The composition of the present invention may be constituted as a seasoning.

Such “other component” is not particularly limited as long as it can be ingested orally. As the “other component”, those used by blending with seasonings, foods, or drinks can be utilized.

Specific examples of the “other component” include excipients such as dextrin, lactose and the like; seasonings such as sodium chloride, sodium glutamate, sodium inosinate, yeast extract, meat extract, seafood extract, vegetable extract, protein hydrolysate, protein partial hydrolysate and the like; proteins such as plant protein, gluten, egg white, gelatin, casein and the like; emulsifiers such as (poly)glycerol fatty acid ester, sorbitan fatty acid ester, sucrose fatty acid ester, propylene glycol ester of fatty acid, calcium stearoyl lactate, various phosphates, lecithin (soybean and egg-yolk), saponin and the like; chelating agents such as citrate, polymerized phosphate and the like; reducing agents such as glutathione, cysteine and the like; lye water; dye; acidulants such as vinegar powder, high acidic vinegar powder, citric acid, succinic acid, lactic acid, malic acid, tartaric acid, gluconic acid, phosphoric acid, citrus juice and the like; flavors such as peppermint flavor, orange flavor, sesame flavor, ginger flavor, garlic flavor and the like; enzymes such as trans glutaminase, lipase and the like; and the like. These components may be used alone, or in any combination.

Examples of the form of the composition of the present invention include solids such as powder and the like, liquid, paste and the like, and any form may be employed as long as addition to meat by uniform penetration or mixing is possible, and the effect of the present invention can be afforded.

The term “powder” refers to an aggregate of particles, and is a concept that includes granules and the like in addition to granules, micro granules, and fine granules and the like, and also includes particle powders and the like obtained by mixing them.

When the composition of the present invention is a solid (powder), the composition may contain only gluconate and a saccharide-modifying enzyme, or may contain the above-mentioned other components. The content of gluconate in the composition is, typically 0.05 to 99.9 wt %, preferably 0.25 to 50 wt %, more preferably 1 to 20 wt %, based on the gluconic acid, to the total amount of the composition from the aspects of qualities such as texture, taste, flavor and the like. The enzyme activity of the saccharide-modifying enzyme in the composition is typically 100 to 100,000,000 U, preferably 500 to 50,000,000 U, more preferably 2,000 to 20,000,000 U, per 1 g of gluconic acid.

When the composition of the present invention is a liquid, a solvent such as water and the like and, where necessary, the above-mentioned other components may be added. The content of gluconate in the composition is typically 0.05 to 99.9 wt %, preferably 0.25 to 50 wt %, more preferably 1 to 20 wt %, based on the gluconic acid, to the total amount of the composition from the aspects of qualities such as texture, taste, flavor and the like. The enzyme activity of the saccharide-modifying enzyme in the composition is typically 100 to 100,000,000 U, preferably 500 to 50,000,000 U, more preferably 2,000 to 20,000,000 U, per 1 g of gluconic acid.

When the composition of the present invention is a paste, an additive to provide a paste (state with flowability and high viscosity) conventionally used for meat processing and, where necessary, the above-mentioned other components may be added. The content of gluconate in the composition is typically 0.05 to 99.9 wt %, preferably 0.25 to 50 wt %, more preferably 1 to 20 wt %, based on the gluconic acid, to the total amount of the composition from the aspects of qualities such as texture, taste, flavor and the like. The enzyme activity of the saccharide-modifying enzyme in the composition is typically 100 to 100,000,000 U, preferably 500 to 50,000,000 U, more preferably 2,000 to 20,000,000 U, per 1 g of gluconic acid.

The composition of the present invention can improve or enhance meat texture after heat cooking by processing the meat as follows:

when the composition is a powder, it is added to meat at a proportion of typically not less than 0.00001 g, preferably not less than 0.0001 g, more preferably not less than 0.001 g, per 100 g of the meat,
when the composition is a liquid, meat is immersed therein at a proportion of typically not less than 10 g, preferably not less than 20 g, more preferably not less than 30 g, per 100 g of the meat,
when the composition is a paste, it is applied to meat at a proportion of typically not less than 0.0001 g, preferably not less than 0.001 g, more preferably not less than 0.01 g, per 100 g of the meat.

The composition of the present invention can be produced by general production techniques for foods and the like and in accordance with the aforementioned components, amounts to be added and forms.

The composition of the present invention may be added at any stage of the production step of a prepared meat product. It is preferable to directly add the composition to raw meat since the effect of the present invention can be obtained easily.

The composition of the present invention may be added only once to meat, or may be added in two or more portions.

The composition of the present invention can be used as a meat modifier when cooking meat by blending with (1) powdered base material such as wheat flour, powder seasoning and the like; (2) liquid seasoning such as broth, sauce and the like; (3) liquid edible fats and oil for cooking such as soybean oil, corn oil and the like; (4) plastic oil such as refined lard, shortening and the like, (5) W/O emulsion composition such as margarine and the like, (6) O/W emulsion composition and the like.

The present invention also contains a production method of a prepared meat product including a step of bringing meat in contact with gluconate and a saccharide-modifying enzyme (hereinafter sometimes to be abbreviated as “the method of the present invention”).

In the present invention, the “prepared meat product” means a food before heating, a material food to be subjected to a cooking treatment such as heating and the like, and a food that has been cooked by heating and the like.

In the method of the present invention, a method for bringing meat in contact with gluconate and a saccharide-modifying enzyme is not particularly limited and attaching gluconate and a saccharide-modifying enzyme to a meat surface, immersing meat therein, coating meat therewith, kneading them into meat (minced meat), introducing them into the inside of meat using an injector, tumbling meat therewith, introducing them during tenderizing and the like can be mentioned.

Examples of the method for bringing into contact with a meat surface include a method of sprinkling on the surface of meat using fine mesh such as colander, sieve and the like, a method of sprinkling directly on the whole meat surface, a method of shaking the composition and meat together in a plastic bag, a method of spreading the composition on a thin tray such as a vat and the like and bringing meat in contact with the composition and the like.

To achieve uniform contact with the meat, gluconate and a saccharide-modifying enzyme may be mixed in advance with a trituration (subsidiary material) and the like and diluted before use. Examples of the trituration include dextrin, sodium chloride, sodium glutamate and the like. The mixing ratio of gluconate and the saccharide-modifying enzyme and the trituration can be appropriately adjusted according to the amounts of gluconate and the saccharide-modifying enzyme to be added.

Examples of the method for immersing meat include a method of preparing a liquid by dissolving or dispersing gluconate and a saccharide-modifying enzyme in a solution at a salt concentration used for conventional meat processing, and immersing the meat therein and the like. Examples of the solution include brine, phosphate solution such as sodium polyphosphate and the like, alkaline solution such as sodium carbonate and the like, starch solution such as potato starch and the like, pickle such as sugar, vinegar, soy sauce and the like, and the like.

Examples of the method for coating meat include a method of mixing gluconate and a saccharide-modifying enzyme with an additive for forming a paste (state with flowability and high viscosity) used for conventional meat processing to prepare a paste and applying same to meat and the like. Examples of the additive include water, emulsifier, starch, polysaccharide thickener, mucopolysaccharide, mannan, fats and oils, fatty acid, glycerol, sugar alcohol and the like.

Among these, a method for immersing meat, kneading into meat (minced meat), or introducing into the inside of meat using injector is preferable, from the aspects of ease of obtaining effects, workability and convenience.

The amount of gluconate to be contacted (added) varies depending on the form of meat. It is typically 0.001 to 5 g, preferably 0.005 to 2.5 g, more preferably 0.01 to 1 g, based on gluconic acid, per 100 g of the meat. When it is within this range, syneresis due to heating can be suppressed and a juicy and good texture after cooking can be maintained.

The amount of a saccharide-modifying enzyme to be contacted (added) varies depending on the form of meat. It is an amount corresponding to the enzyme activity of typically 0.1 to 500,000,000 U, preferably 0.5 to 250,000,000 U, more preferably 1 to 100,000,000 U. When it is within this range, syneresis due to heating can be suppressed and a juicy and good texture after cooking can be maintained.

The proportion of gluconate and a saccharide-modifying enzyme to be contacted in the method of the present invention is typically 100 to 100,000,000 U, preferably 500 to 50,000,000 U, more preferably 2,000 to 20,000,000 U, of a saccharide-modifying enzyme per 1 g of gluconic acid.

The time during which the composition of the present invention is contacted with meat (time after addition of the composition of the present invention to meat and before being subjected to cooking, packaging, freezing step, and the like) is not particularly limited as long as gluconate and a saccharide-modifying enzyme can act on the meat. In view of ease of obtaining effects and handling at meat processing sites, it is typically not less than 30 min, preferably not less than 60 min, more preferably not less than 120 min, further preferably not less than 180 min, particularly preferably not less than 18 hr. While the upper limit is not particularly set, it is typically not more than 72 hr, preferably not more than 20 hr.

The contact temperature (reaction temperature) is not particularly limited as long as the enzyme can maintain activity. From the aspect of reaction efficiency, it is typically 0 to 60° C., preferably 0 to 30° C. When the retention time is not less than 1 hr, from the aspect of food hygiene, it is preferable to keep the meat in a refrigerator.

The pH at the time of contact is not particularly limited as long as the enzyme maintains the activity. From the aspect of the general pH range of the food to be eaten, it is typically pH 3 to 10, preferably pH 5 to 8.

The method of the present invention may appropriately contain conventional processes such as a step of cutting meat, a step of mixing meat, and a step of adding other seasonings and additives.

The present invention also includes a method for modifying meat including a step of contacting meat with gluconate and a saccharide-modifying enzyme.

The definition, contact amount, preferable range and the like of each component are as described above.

A prepared meat product produced by the method of the present invention is also included in the present invention.

Specifically, the prepared meat product of the present invention is a cooked product containing added gluconate in an amount of typically 0.001 to 5 g, preferably 0.005 to 2.5 g, more preferably 0.01 to 1 g, based on gluconic acid, and added saccharide-modifying enzyme in an amount corresponding to typically 0.1 to 500,000,000 U, preferably 0.5 to 250,000,000 U, more preferably 1 to 100,000,000 U, per 100 g of meat.

Various definitions, preferable range and the like are as described above.

Other features of the invention will become apparent in the course of the following descriptions of exemplary embodiments which are given for illustration of the invention and are not intended to be limiting thereof.

### EXAMPLES

Unless otherwise specified, the sensory evaluation in this Example was performed using a sufficiently-trained expert panel who has been engaged in food business for not less than 5 years. In the present specification, % means % by weight unless otherwise specified.

### Example 1 Evaluation of Water-Holding Property of Meat

### 1. Preparation Method of Sample

(1) Skin and fat were removed from the breast meat of commercially available domestic broiler chicken.
(2) The meat was formed into a 30 mm long×50 mm wide×5 mm thick cuboid (so that the side is parallel to the muscle fiber direction).
(3) Various materials shown in Table 1 were dissolved in a 0.3 M NaCl aqueous solution (salt concentration used for general meat processing; sodium chloride used was “Nakuru four 2” (manufactured by Naikai Salt Industries Co., Ltd.)) and used as sample solutions.
(4) The obtained sample solution was added to the reshaped chicken breast meat at 20%[w/w] based on the meat weight.
(5) After measurement of weight, this was vacuum packaged using a vacuum packaging machine “V-455G-1” (manufactured by TOSEI).
(6) The package was stored at 4° C. overnight (for 18 hr).
(7) The treated meat was stood at room temperature for 20 min and returned to ordinary temperature.
(8) Using a thermostatic tank “Thermal Robo TR-1AR” (manufactured by AS ONE), the meat was heated in water bath at 75° C. for 30 min.
(9) The heated sample was taken out from a vacuum pack, water on the surface was wiped off with a paper towel and the weight was measured.
(10) From the weight difference before and after heating, the syneresis rate was determined by the following equation.

Syneresis (%)=((weight of separated water)/(weight before heating))×100

where the weight of separated water=weight before heating−weight after heating

The syneresis rate indicates the proportion of water lost by heating when the water held by the meat before heating is 100%. A lower value indicates a higher water-holding effect.

### 2. Evaluation of Syneresis Rate

The results of the syneresis rate measurement are based on the syneresis rate of about 30% in the non-addition plot (test plot No. 1) as the standard and are as follows:

“−”: no effect or adverse effect with syneresis rate of not less than 30%
“+”: water-holding effect with syneresis rate of not less than 25% and less than 30%
“++”: high water-holding effect with syneresis rate of not less than 20% and less than 25%
“+++”: remarkable water-holding effect with syneresis rate of less than 20%.

The results are shown in the following Table 1.

TABLE 1

| | | | amount added | | |
|---|---|---|---|---|---|
| | | | | amount of | |
| | | | amount | enzyme | |
| test | | | added [g] | added [U] | syneresis rate |
| plot | | | per 100 g | per 100 g | measurement |
| No. | category | test plot | of meat | of meat | results |
| 1 | — | — | — | — | − |
| 2 | organic | sodium citrate | 0.085 | — | − |
| 3 | acid salt | calcium lactate | 0.10 | — | − |
| 4 | | monosodium fumarate | 0.16 | — | + |
| 5 | | sodium malate | 0.12 | — | − |
| 6 | | sodium gluconate | 0.10 | — | ++ |
| 7 | saccharide- | glucoamylase | — | 1875 | − |
| 8 | modifying | α-glucosidase | — | | − |
| | enzyme | | | | |
| 9 | combined | sodium | sodium | | +++ |
| | use | gluconate × | gluconate | | |
| | | glucoamylase | 0.10 | | |
| 10 | | sodium | sodium | | +++ |
| | | gluconate × | gluconate | | |
| | | α-glucosidase | 0.10 | | |

Organic acid salts of test plot Nos. 2 to 6 were added such that the same mol concentration was achieved at the time of immersion.

Glucoamylase and α-glucosidase were used in the general amounts used for foods.

As the glucoamylase for test plot Nos. 7 and 9, “glucoamylase for sake brewing “AMANO” SD” (manufactured by Amano Enzyme) was used concurrently with 0.6% potato starch solution.

As the α-glucosidase for test plot Nos. 8 and 10, “α-glucosidase “AMANO”” (manufactured by Amano Enzyme) was used concurrently with 0.6% potato starch solution.

As shown in Table 1, a high water-holding effect was confirmed with sodium gluconate, sodium gluconate×glucoamylase and sodium gluconate×α-glucosidase. Particularly, a synergistic water-holding effect was confirmed with sodium gluconate×glucoamylase and sodium gluconate×α-glucosidase.

### Example 2 Evaluation of Water-Holding Property of Meat

### 1. Preparation Method of Sample

Samples were prepared by a method similar to that in Example 1 except that various materials shown in Table 2 were used, and the syneresis rate was determined by a similar method.

### 2. Evaluation of Syneresis Rate

The syneresis rate was evaluated based on the same standard as in Example 1.

The results are shown in the following Table 2.

TABLE 2

| | | | amount added | | |
|---|---|---|---|---|---|
| | | | amount of | | |
| | | | organic | amount of | |
| | | | acid salt | enzyme | |
| test | | | added [g] | added [U] | syneresis rate |
| plot | | | per 100 g | per 100 g | measurement |
| No. | category | test plot | of meat | of meat | results |
| 1A | — | — | — | — | − |
| 2A | saccharide- | glucoamylase | — | 1875 | − |
| | modifying | | | | |
| | enzyme | | | | |
| 3A | combined | sodium | 0.1 | | − |
| | use | citrate × | | | |
| | | glucoamylase | | | |
| 4A | | potassium | | | − |
| | | citrate × | | | |
| | | glucoamylase | | | |
| 5A | | calcium | | | − |
| | | citrate × | | | |
| | | glucoamylase | | | |
| 6A | | sodium | | | +++ |
| | | gluconate × | | | |
| | | glucoamylase | | | |
| 7A | | potassium | | | +++ |
| | | gluconate × | | | |
| | | glucoamylase | | | |
| 8A | | calcium | | | ++ |
| | | gluconate × | | | |
| | | glucoamylase | | | |

As glucoamylase for test plot Nos. 2A to 8A, “glucoamylase AMANO SD” (manufactured by Amano Enzyme) was used in the general amounts used for foods.

Glucoamylase for test plot Nos. 2A to 8A was used concurrently with 0.6% potato starch solution.

As sodium/potassium/calcium citrate of test plot Nos. 3A to 5A, “purified sodium citrate”, “purified potassium citrate”, “calcium citrate” (manufactured by Fuso Chemical Co., Ltd.) were respectively used.

As sodium/potassium/calcium gluconate of test plot Nos. 6A to 8A, “Herushasu A”/“Herushasu K”/“calcium gluconate” (manufactured by Fuso Chemical Co., Ltd.) were respectively used.

As shown in Table 2, gluconate (sodium salt, potassium salt, calcium salt)×glucoamylase showed a high water-holding effect in combination with any salts. On the other hand, an organic acid salt, citrate (sodium salt, potassium salt, calcium salt) which is a general food additive×glucoamylase and glucoamylase alone, did not show a water-holding effect.

### Example 3 Sensory Evaluation of Meat Modifying Effect

### 1. Preparation of Samples (Minced Meat (Frozen Dumplings))

(1) The test plot materials shown in the following Table 4 were added to the starting materials shown in the following Table 3.
(2) Using a kitchen aid mixer (manufactured by FMI), the materials were mixed at set speed 1 for 3 min to give a dumpling filling.
(3) 15 g of dumpling filling was wrapped with a commercially available dumpling skin.
(4) Dumplings were heated at 100° C. for 2.5 min (core temperature 67° C.) in a steam convection oven (manufactured by Fujimac).
(5) The dumplings were rapidly frozen at −30° C. in a blast chiller (manufactured by Fukushima Kogyo).
(6) Six dumplings were packaged by a vacuum packing machine (manufactured by TOSEI).
(7) The packaged gyoza was frozen and stored for 1 hr.
(8) City water (80 mL) was added to 12 gyoza.
(9) After steaming for 5 min on a hot plate (manufactured by ZOJIRUSHI) at 230° C., the lid was removed and gyoza was baked for 1 min.
(10) The obtained fried gyoza was subjected to sensory evaluation.

TABLE 3

| starting material | composition [%] |
|---|---|
| minced pork | 18 |
| lard | 9 |
| egg | 4 |
| vegetable category (onion, | 50 |
| cabbage, garlic, chive) | |
| seasoning category (sugar, | 14 |
| salt, sesame oil, etc.) | |
| water | 5 |
| total | 100 |

### 2. Sensory Evaluation of Ground Meat (Frozen Gyoza)

The sensory evaluation was performed for juicy feeling (juicy feeling during mastication) by a panel of 4 experts, and a no-addition plot (test plot No. 1) was used as a control. The sensory evaluation results are as follows:

“−”: no effect or adverse effect
“+”: effective
“++”: remarkable effect
“+++”: very remarkable effect.

The results are shown in the following Table 4.

TABLE 4

| | | | amount added | | |
|---|---|---|---|---|---|
| | | | | amount of | |
| | | | amount | enzyme | sensory |
| | | | added [g] | added [U] | evaluation |
| test | | | per 100 g | per 100 g | results |
| plot | | | of minced | of minced | juicy |
| No. | category | test plot | meat | meat | feeling |
| 1B | — | — | — | — | − |
| 2B | general | starch × | starch 16 | — | + |
| | juicy | dextrin | dextrin 8 | | |
| | feeling | | | | |
| | improving | | | | |
| | material | | | | |
| 3B | the | sodium | sodium | 1250 | +++ |
| | present | gluconate × | gluconate | | |
| | material | glucoamylase | 0.1 | | |

As the starch of test plot No. 2B, “potato starch AH-5” (manufactured by Matsutani Chemical Industry Co., Ltd.) was used.

As the dextrin of test plot No. 2B, “Paindex #1” (manufactured by Matsutani Chemical Industry Co., Ltd.) was used.

As the glucoamylase of test plot No. 3B, “glucoamylase AMANO SD” (manufactured by Amano Enzyme) was used.

A very remarkable improving effect on the juicy feeling was confirmed in the combination of sodium gluconate and glucoamylase rather than in the combination of starch and dextrin, which is generally used for improving the juicy feeling.

### INDUSTRIAL APPLICABILITY

According to the present invention, meat that does not lose umami or juiciness of the meat even after heating and can afford a juicy and tender texture can be provided.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition for modifying meat, comprising at least one gluconate and at least one saccharide-modifying enzyme.

2. The composition according to claim 1, wherein said at least one saccharide-modifying enzyme is present in an amount of 100 to 100,000,000 U per 1 g of gluconic acid.

3. The composition according to claim 1, wherein said at least one saccharide-modifying enzyme is glucoamylase or α-glucosidase.

4. The composition according to claim 1, wherein said at least one gluconate is at least one member selected from the group consisting of sodium gluconate, potassium gluconate, and calcium gluconate.

5. A method for producing a prepared meat product, comprising contacting meat with at least one gluconate and at least one saccharide-modifying enzyme.

6. The method according to claim 5, wherein said at least one saccharide-modifying enzyme is glucoamylase or α-glucosidase.

7. The method according to claim 5, comprising contacting said meat with 0.001 to 5 g of said at least one gluconate, based on gluconic acid, per 100 g of the meat.

8. The method according to claim 5, comprising contacting said meat with 0.1 to 500,000,000 U of said at least one saccharide-modifying enzyme per 100 g of the meat.

9. A prepared meat product, comprising added gluconate and added saccharide-modifying enzyme, wherein said added gluconate is present in an amount of 0.001 to 5 g of gluconic acid and said added saccharide-modifying enzyme is present in an amount of 0.1 to 500,000,000 U, each per 100 g of meat.

10. A method for modifying meat, comprising contacting meat with at least one gluconate and at least one saccharide-modifying enzyme.
